(19) United States
(12) Patent Application Publication
(10) Pub. No.: US 2009/0117640 A1
(43) Pub. Date: May 7, 2009

(54) Transglutaminase Variants with Improved Specificity

(75) Inventors: Leif Norskov-Lauritsen, Tapernoje (DK); Nils Langeland Johansen, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: novo Nordisk Health Care AG, Zurich (CH)

(21) Appl. No.: 12/063,693

(22) PCT Filed: Aug. 18, 2006
(86) PCT No.: PCT/EP2006/065439

§ 371 Date: Jan. 23, 2009

(30) Foreign Application Priority Data
Aug. 18, 2005 (EP) 05107599.2

(51) Int. Cl. C12N 9/10; C12N 15/11; C12N 15/00; C12N 1/21; C12N 1/19

(52) U.S. Cl. 435/193; 536/23.2; 435/320.1; 435/252.3; 435/254.2

(57) ABSTRACT

Variants of transglutaminase from S. mobareanse, which variants have improved selectivity against Gln-40 og Gln-141 of human growth hormone are provided.

### FIELD OF THE INVENTION

The present invention relates to novel variants of transglutaminase from Streptomyces mobareanse. The variant can be used for modifying peptides with improved selectivity.

### BACKGROUND OF THE INVENTION

It is well-known to modify the properties and characteristics of peptides by conjugating groups to said proteins which duly changes the properties. In particular for therapeutic peptides it may desirable or even necessary to conjugate groups to said peptides which prolong the half life of the peptides. Typically such conjugating groups are polyethylene glycol (PEG) or fatty acids—see J. Biol. Chem. 271, 21969-21977 (1996).

Transglutaminase (TGase) has previously been used to alter the properties of peptides. In the food industry and particular in the diary industry many techniques are available to e.g. cross-bind peptides using TGase. Other documents disclose the use of TGase to alter the properties of physiologically active peptides. EP 950665, EP 785276 and Sato, Adv. Drug Delivery Rev. 54, 487-504 (2002) disclose the direct reaction between peptides comprising at least one Gin and amine-functionalised PEG or similar ligands in the presence of TGase, and Wada in Biotech. Lett. 23, 1367-1372 (2001) discloses the direct conjugation of β-lactoglobulin with fatty acids by means of TGase. The international patent application WO2005070468 discloses that TGase may be used to incorporate a functional group into a glutamine containing peptide to form a functionalised peptide, and that this functionalised peptide in a subsequent step may be reacted with e.g. a PEG capable of reacting with said functionalised protein to form a PEGylated peptide.

Transglutaminase (E.C.2.3.2.13) is also known as protein-glutamine-γ-glutamyltransferase and catalyses the general reaction

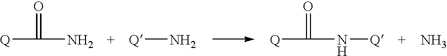

wherein Q-C(O)—NH₂ may represent a glutamine containing peptide and Q′-NH₂ then represents an amine donor providing the functional group to be incorporated in the peptide in the reaction discussed above.

A common amine donor in vivo is peptide bound lysine, and the above reaction then affords cross-bonding of peptides. The coagulation factor Factor XIII is a transglutaminase which effects clotting of blood upon injuries. Different TGase's differ from each other, e.g. in what amino acid residues around the Gin are required for the protein to be a substrate, i.e. different TGase's will have different Gln-containing peptides as substrates depending on what amino acid residues are neighbours to the Gln residue. This aspect can be exploited if a peptide to be modified contains more than one Gln residue. If it is desired to selectively conjugate the peptide only at some of the Gln residues present this selectivity can be obtained be selection of a TGase which only accepts the relevant Gln residue(s) as substrate.

Human growth hormone (hGH) comprises 11 glutamine residues, and any TGase mediated conjugation of hGH is thus potentially hampered by a low selectivity. It has been found that under certain reaction conditions, the two step conjugation reaction described above, wherein hGH is functionalised in a S. mobaraense TGase mediated reaction, may give rise to hGH which has been functionalised at two positions, i.e. 40-Gln and 141-Gln. There is a need for identifying variants of TGase which mediates a more specific functionalization of hGH.

### SUMMARY OF THE INVENTION

The present inventor has surprisingly found that the substitution of certain amino acid residues in TGase from S. mobaraense affords a TGase which mediates a more specific functionalization of hGH.

In one embodiment, the invention relates to a TGase from S. mobaraense (SEQ ID No. 1) wherein up to three acid or basic amino acid residues have been substituted with other basic or acidic amino acid residues.

In one embodiment, the invention relates to an isolated peptide comprising an amino acid sequence having at least 80% identity with the amino acid sequence of the TGase from S. mobaraense, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

In one embodiment, the invention relates to a TGase from S. mobaraense (SEQ ID No. 1), wherein said sequence is modified in one or more of the amino acid residues selected from Asp-4, Val-30, Tyr-75, Arg-89, Glu-115, Ser-210 Asp-221, Ala-226 Pro-227, Gly-250, Tyr-302, Asp-304, and Lys-327.

In one embodiment, the invention relates to a peptide as defined in SEQ ID No. 1 comprising one or more of the substitutions Tyr-75→acidic amino acid residue; Tyr-302→basic amino acid residue; and Asp-304→basic amino acid residue.

In one embodiment, the invention relates to a nucleic acid construct encoding a peptide according to the present invention.

In one embodiment, the invention relates to a vector comprising a nucleic acid encoding a peptide according to the present invention.

In one embodiment, the invention relates to a host comprising a a vector comprising a nucleic acid encoding a peptide according to the present invention.

In one embodiment, the invention relates to a composition comprising a peptide according to the present invention.

In one embodiment, the invention relates to a method of conjugating hGH, the method comprising reacting hGH with an amine donor in the presence of a peptide according to the present invention.

### BRIEF DESCRIPTION OF THE FIGURES

FIG. 1 shows a sequence alignment of the sequence of the above mentioned TGase from Streptomyces mobaraensis and the above mentioned TGase from Streptoverticillium ladakanum.

FIG. 2 shows a picture of a typical CE analysis of a TGase-catalyzed transglutamination of hGH with 1,3-diamino-2-propanol.

FIG. 3. Analysis of reaction mixture of hGH mutants catalyzed by S. ladakanum TGase by HPLC. Top: hGH-Q40N. The first peak (26.5 min, area 1238) is product-Q141 and the second peak (29.7 min, area 375) is the remaining hGH-Q40N. Bottom: hGH-Q141N. The first peak (19.2 min, area 127) is product-Q40 and the second peak (30.3 min, area 1158) is the remaining hGH-Q141N.

FIG. 4. Analysis of reaction mixture of hGH mutants catalyzed by S. mobarense TGase by HPLC. Top: hGH-Q40N. The first peak (26.9 min, area 1283) is product-Q141 and the second peak (30.1 min, area 519) is the remaining hGH-Q40N. Bottom: hGH-Q141N. The first peak (19.5 min, area 296) is product-Q40 and the second peak (30.6 min, area 1291) is the remaining hGH-Q141N.

### DESCRIPTION OF THE INVENTION

In the present context, the term “acidic amino acid residue” is intended to indicate a natural amino acid residue with a pKa below 7. Particular examples include Asp and Glu.

In the present context, the term “basic amino acid residue” is intended to indicate a natural amino acid residue with a pKa above 7. Particular examples include Tyr, Lys and Arg.

In the present context “transamination” or similar is intended to indicate a reaction where nitrogen in the side chain of glutamine is exchanged with nitrogen from another compound, in particular nitrogen from another nitrogen containing nucleophile.

The term “conjugate” as a noun is intended to indicate a modified peptide, i.e. a peptide with a moiety bonded to it to modify the properties of said peptide. As a verb, the term is intended to indicate the process of bonding a moiety to a peptide to modify the properties of said peptide.

In the present context, the terms “specificity” and “selectivity” are used interchangeably to describe a preference of the TGase for reacting with one or more specific glutamine residues in hGH as compared to other specific glutamine residues in hGH. For the purpose of this specification, the specificity of the peptides of the invention for Gln-40 as compared to Gln141 in hGH are decided according to the results of testing the peptides as described in Example 3.

The micro-organism Streptomyces mobaraensis is also classified as Streptoverticillium mobaraense. A TGase may be isolated from the organism, and this TGase is characterised by a relatively low molecular weight (˜38 kDa) and by being calcium-independent. The TGase from S. mobaraense is relatively well-described; for instance has the crystal structure been solved (US 156956; Appl. Microbiol. Biotech. 64, 447-454 (2004)).

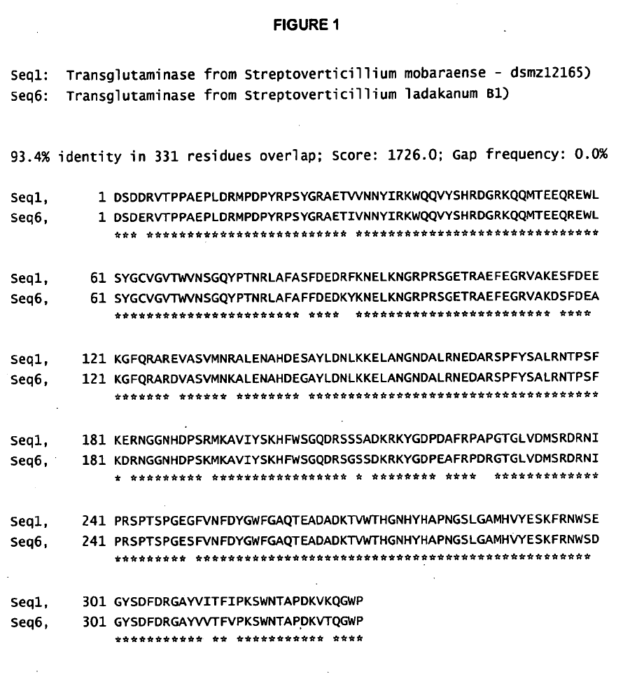

The sequence of a TGase isolated from Streptoverticillium ladakanum has an amino acid sequence which is identical to the sequence from Streptoverticillium mobaraense except for a total of 22 amino acid differences between the two sequences (Yi-Sin Lin et al., Process Biochemistry 39(5), 591-598 (2004). The sequence of the TGase from Streptomyces mobaraensis is given in SEQ ID No. 1, the sequence of the TGase from Streptomyces mobaraensis is given in SEQ ID No. 6, and FIG. 1 shows a sequence alignment of the sequence of the above mentioned TGase from Streptomyces mobaraensis and the above mentioned TGase from Streptoverticillium ladakanum.

One way of preparing conjugated hGH comprises a first reaction between hGH and an amine donor comprising a functional group to afford a functionalised hGH, said first reaction being mediated (i.e. catalysed) by a TGase. In a second reaction step, said functionalised hGH is further reacted with e.g. a PEG or fatty acid capable or reacting with said incorporated functional group to provide conjugated hGH. The first reaction is sketched below.

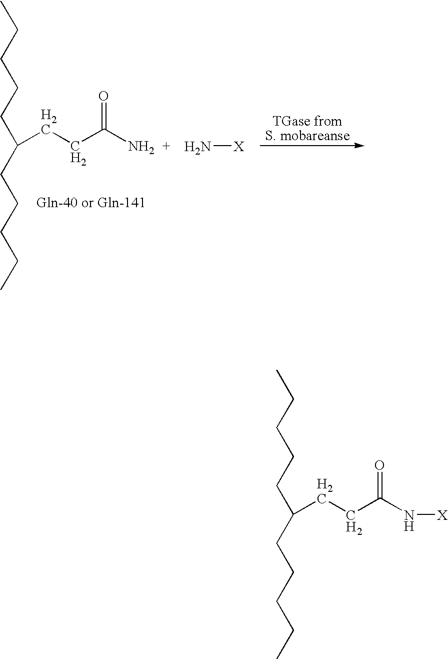

X represent a functional group or a latent functional group, i.e. a group which upon further reaction, e.g. oxidation or hydrolysation is transformed into a functional group.

When the reaction above is mediated by TGase from S. mobaraense the reaction between hGH and H₂N—X (the amine donor) takes place predominately at Gln-40 and Gln-141. The above reaction may be employed to e.g. PEGylate hGH to achieve a therapeutic growth hormone product with a prolonged half life. As it is generally held desirable that therapeutic-compositions are single-compound compositions, the above discussed lack of specificity requires a further purification step wherein Gln-40 functionalised hGH is separated from Gln-141 functionalised and/or Gln-40/Gln-141 double-functionalised hGH.

Such use of transglutaminase for conjugations of human growth hormone is extensively described in WO2005/070468 and WO2006EP063246.

The peptides of the present invention have a specificity for Gln-40 compared to Gln-141 of hGH, which is different from the specificity of a peptide having an amino acid sequence as shown in SEQ ID No. 1 for Gln-40 compared to Gln-141 measured as describing in Example 3. Peptides of the present invention may thus be used in a method for transglutaminating hGH to increase production of Gln-40 functionalised hGH or Gln-141 functionalised hGH as compared to a reaction using a TGase having the amino acid sequence of SEQ ID No. 1.

In one embodiment, the present invention provides an isolated peptide comprising an amino acid sequence having at least 80% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, add Lys-327.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48,1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, a peptide of the present invention comprises an amino acid sequence as defined in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, og Leu-285, Tyr-302, Asp-304, and Lys-327.

In one embodiment, a peptide of the present invention comprises an amino acid sequence as defined in SEQ ID No. 1, wherein said sequence is modified in one or more of the amino acid residues situated less than 20 Å away from Cys-64, such as for instance less than 15 Å away from Cys-64.

The distance from Cys64 is measured using the crystal structure of the TGase from S. mobaraense as published in Tatsuki Kashiwagi et al., Journal of Biological Chemistry 277(46), 44252-44260 (2002). The atom coordinates are also deposited under in Protein Databank as code 1IU4.

Examples of amino acids in TGase from S. mobaraense situated less than 15 Å away from Cys64 include the following:

Arg5, Val6, Thr7, Glu28, Thr29, Val30, Val31, Tyr34, Gln56, Arg57, Glu58, Trp59, Leu60, Ser61, Tyr62, Gly63, Val65, Gly66, Val67, Thr68, Trp69, Val70, Asn71, Ser72, Gln74, Tyr75, Pro76, Thr77, Asn78, Tyr198, Ser 99, Lys200, His201, Phe202, Trp203, Asn239, Ile240, Pro241, Phe251, Val252, Asn253, Phe254, Asp255, Tyr256, Gly257, Trp258, Phe259, Trp272, Thr273, His274, Gly275, Asn276, His277, Tyr278, His279, Ala280, Leu285, Gly286, Ala287, Met288, His289, Val290, Tyr291, Glu292, Ser293, Asn297, Trp298, Ser299, Gly301, Tyr302, Asp304, Phe305, Gly308, and Ala309.

In one embodiment, a peptide of the present invention comprises an amino acid sequence as defined in SEQ ID No. 1, wherein said sequence is modified in one or more of the amino acid residues selected from Asp-4, Arg-89, Glu-115, Ser-210, Asp-221, and Lys-327.

In one embodiment, a peptide of the present invention comprises an amino acid sequence as defined in SEQ ID No. 1, wherein said sequence is modified in one or more of the amino acid residues selected from Ala-226 and Pro-227.

In one embodiment, a peptide of the present invention comprises an amino acid sequence as defined in SEQ ID No. 1, wherein said sequence is modified in Tyr-75.

In one embodiment, a peptide of the present invention comprises an amino acid sequence as defined in SEQ ID No. 1, wherein said sequence is modified in Tyr-302.

In one embodiment, a peptide of the present invention comprises an amino acid sequence as defined in SEQ ID No. 1, wherein said sequence is modified in Asp-304.

In one embodiment, a peptide of the present invention comprises an amino acid sequence as defined in SEQ ID No. 1, wherein said sequence is as defined in SEQ ID No. 6

In one embodiment, a peptide of the present invention has a specificity for Gln-40 of hGH compared to Gln-141 of hGH, which is different from the specificity of a peptide having an amino acid sequence as shown in SEQ ID No. 1 for Gln-40 of hGH compared to Gln-141 of hGH.

In one embodiment, the specificity of a peptide of the present invention for Gln-40 compared to Gln-141 is higher than the specificity of a peptide having an amino acid sequence as shown in SEQ ID No. 1 for Gln-40 compared to Gln-141, which results in an increase in the production of Gln-40 as compared to Gln-141 in a transglutaminase reaction using TGase as described herein.

In one embodiment, the specificity for a peptide of the present invention for Gln-40 compared to Gln-141 is at least 1.25, such as at least 1.50, for instance at least 1.75, such as at least 2.0, for instance at least 2.5, such as at least 3.0, for instance at least 3.5, such as at least 4.0, for instance at least 4.5, such as at least 5.0, for instance at least 5.5, such as at least 6.0, for instance at least 6.5, such as at least 7.0, for instance at least 7.5, such as at least 8.0, for instance at least 8.5, such as at least 9.0, for instance at least 9.5, such as at least 10.0 times higher than the specificity of a peptide having an amino acid sequence as shown in SEQ ID No. 1 for Gln-40 compared to Gln-141.

In one embodiment, the specificity of a peptide of the present invention for Gln-141 compared to Gln-40 is higher than the specificity of a peptide having an amino acid sequence as shown in SEQ ID No. 1 for Gln-141 compared to Gln-40, which results in an increase in the production of Gln-141 as compared to Gln-40 in a transglutaminase reaction using TGase as described herein.

In one embodiment, the specificity for a peptide of the present invention for Gln-141 compared to Gln-40 is at least 1.25, such as at least 1.50, for instance at least 1.75, such as at least 2.0, for instance at least 2.5, such as at least 3.0, for instance at least 3.5, such as at least 4.0, for instance at least 4.5, such as at least 5.0, for instance at least 5.5, such as at least 6.0, for instance at least 6.5, such as at least 7.0, for instance at least 7.5, such as at least 8.0, for instance at least 8.5, such as at least 9.0, for instance at least 9.5, such as at least 10.0 times higher than the specificity of a peptide having an amino acid sequence as shown in SEQ ID No. 1 for Gln-141 compared to Gln-40.

In one embodiment, the present invention provides a transglutaminase peptide having a specificity for Gln-40 of hGH compared to Gln-141 of hGH, which is different from the specificity of a peptide having an amino acid sequence as shown in SEQ ID No. 1 for Gln-40 of hGH compared to Gln-141 of hGH.

In one embodiment, the present invention provides a transglutaminase peptide having a specificity for Gln-40 of hGH compared to Gln-141 of hGH, which is higher than the specificity of a peptide having an amino acid sequence as shown in SEQ ID No. 1 for Gln40 of hGH compared to Gln-141 of hGH.

In one embodiment, the present invention provides a transglutaminase peptide having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

In one embodiment, the present invention provides a nucleic acid construct encoding a peptide according to the present invention.

In one embodiment, the present invention provides a vector comprising the nucleic acid construct according to the present invention.

In one embodiment, the present invention provides a host comprising the vector according to the present invention.

In one embodiment, the present invention provides a composition comprising a peptide according to the present invention.

In one embodiment, the present invention provides a method for conjugating hGH, wherein said method comprises reacting said hGH with an amine donor in the presence of a peptide according to the present invention.

In one embodiment, the present invention provides the use of a peptide according to the present invention in the preparation of a conjugated hGH.

The following is a non-limiting list of embodiments of the present invention:

### EMBODIMENT 1

An isolated peptide comprising an amino acid sequence having at least 80% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

### EMBODIMENT 2

An isolated peptide according to embodiment 1 comprising an amino acid sequence having at least 85% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

### EMBODIMENT 3

An isolated peptide according to embodiment 2 comprising an amino acid sequence having at least 90% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

### EMBODIMENT 4

An isolated peptide according to embodiment 3 comprising an amino acid sequence having at least 95% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

### EMBODIMENT 5

An isolated peptide according to embodiment 4 comprising an amino acid sequence as defined in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

### EMBODIMENT 6

An isolated peptide according to any of embodiments 1 to 5, wherein said sequence is modified in Gly-250.

### EMBODIMENT 7

An isolated peptide according to embodiment 6, wherein Gly-250 is substituted with a Thr.

### EMBODIMENT 8

An isolated peptide according to embodiment 6, wherein Gly-250 is substituted with a Ser.

### EMBODIMENT 9

An isolated peptide according to any of embodiments 1 to 8, wherein said sequence is modified in one or more of the amino acid residues situated less than 20 Å away from Cys-64.

### EMBODIMENT 10

An isolated peptide according to any of embodiments 1 to 9, wherein said sequence is modified in one or more of the amino acid residues situated less than 15 Å away from Cys-64.

### EMBODIMENT 11

An isolated peptide according to embodiment 10, wherein the sequence is not modified in position Cys64.

### EMBODIMENT 12

An isolated peptide according to embodiment 10 or 11, wherein said sequence is modified in one or more of the amino acid residues selected from Val-30, Tyr-62, Val-252, Asn-253, Phe-254, His-277, Tyr-278, and Leu-285.

### EMBODIMENT 13

An isolated peptide according to embodiment 12, wherein said sequence is modified in Tyr-62.

### EMBODIMENT 14

An isolated peptide according to embodiment 12 or embodiment 13, wherein said sequence is modified in His-277 and/or Tyr-278.

### EMBODIMENT 15

An isolated peptide according to any of embodiments 12 to 14, wherein said sequence is modified in Leu-285.

### EMBODIMENT 16

An isolated peptide according to any of embodiments 12 to 15, wherein said sequence is modified in one or more of the amino acid residues selected from Val-252, Asn-253, and Phe-254.

### EMBODIMENT 17

An isolated peptide according to any of embodiments 10 to 16, wherein said sequence is modified in Val-30.

### EMBODIMENT 18

An isolated peptide according to embodiment 17, wherein Val-30 is substituted with an Ile.

### EMBODIMENT 19

An isolated peptide according to any of embodiments 1 to 18, wherein said sequence is modified in one or more of the amino acid residues selected from Asp-4, Arg-89, Glu-115, Ser-210, Asp-221, and Lys-327.

### EMBODIMENT 20

An isolated peptide according to embodiment 19, wherein Asp-4 is substituted with an Glu.

### EMBODIMENT 21

An isolated peptide according to embodiment 19 or embodiment 20, wherein Asp-4 is substituted with an Glu and the amino acids in positions 1, 2 and 3 have been deleted.

### EMBODIMENT 22

An isolated peptide according to any of embodiments 19 to 21, wherein Arg-89 is substituted with a Lys.

### EMBODIMENT 23

An isolated peptide according to any of embodiments 19 to 22, wherein Glu-115 is substituted with an Asp.

### EMBODIMENT 24

An isolated peptide according to any of embodiments 19 to 23, wherein Ser-210 is substituted with a Gly.

### EMBODIMENT 25

An isolated peptide according to any of embodiments 19 to 24, wherein Asp-221 is substituted with a Ser.

### EMBODIMENT 26

An isolated peptide according to any of embodiments 19 to 25, wherein Lys-327 is substituted with a Thr.

### EMBODIMENT 27

An isolated peptide according to any of embodiments 1 to 26, wherein said sequence is modified in one or more of the amino acid residues selected from Ala-226 and Pro-227.

### EMBODIMENT 28

An isolated peptide according to embodiment 27, wherein Ala-226 is substituted with an Asp.

### EMBODIMENT 29

An isolated peptide according to embodiment 27, wherein Pro-227 is substituted with an Arg.

### EMBODIMENT 30

An isolated peptide according to any of embodiments 1 to 29, wherein said sequence is modified in Tyr-75.

### EMBODIMENT 31

An isolated peptide according to embodiment 30, wherein Tyr-75 is substituted with an amino acid different from Glu.

### EMBODIMENT 32

An isolated peptide according to embodiment 31, wherein Tyr-75 is substituted with an amino acid different from Asp or Glu.

### EMBODIMENT 33

An isolated peptide according to embodiment 32, wherein Tyr-75 is substituted with an amino acid different from an acidic amino acid residue.

### EMBODIMENT 34

An isolated peptide according to embodiment 30, wherein Tyr-75 is substituted with an acidic amino acid residue.

### EMBODIMENT 35

An isolated peptide according to embodiment 34, wherein Tyr-75 is substituted with Asp or Glu.

### EMBODIMENT 36

An isolated peptide according to embodiment 35, wherein Tyr-75 is substituted with Glu.

### EMBODIMENT 37

An isolated peptide according to any of embodiments 1 to 36, wherein said sequence is modified in Tyr-302.

### EMBODIMENT 38

An isolated peptide according to embodiment 37, wherein Tyr-302 is substituted with a basic amino acid residue different from Tyr.

### EMBODIMENT 39

An isolated peptide according to embodiment 38, wherein Tyr-302 is substituted with Arg or Lys.

### EMBODIMENT 40

An isolated peptide according to embodiment 39, wherein Tyr-302 is substituted with Arg.

### EMBODIMENT 41

An isolated peptide according to any of embodiments 1 to 40, wherein said sequence is modified in Asp-304.

### EMBODIMENT 42

An isolated peptide according to embodiment 41, wherein Asp-304 is substituted with a basic amino acid residue.

### EMBODIMENT 43

An isolated peptide according to embodiment 42, wherein Asp-304 is substituted with Tyr, Lys or Arg.

### EMBODIMENT 44

An isolated peptide according to embodiment 43, wherein Asp-304 is substituted with Lys.

### EMBODIMENT 45

An isolated peptide according to embodiment 36 having a sequence as defined in SEQ ID No. 2.

### EMBODIMENT 46

An isolated peptide according to embodiment 40 having a sequence as defined in SEQ ID No. 3.

### EMBODIMENT 47

An isolated peptide according to embodiment 44 having a sequence as defined in SEQ ID No. 4.

### EMBODIMENT 48

An isolated peptide according to any of embodiments 30 to 44 having a sequence as defined in SEQ ID No. 5.

### EMBODIMENT 49

An isolated peptide comprising an amino acid sequence having at least 80% identity with the amino acid sequence in SEQ ID No. 6.

### EMBODIMENT 50

An isolated peptide according to embodiment 49 comprising an amino acid sequence having at least 85% identity with the amino acid sequence in SEQ ID No. 6.

### EMBODIMENT 51

An isolated peptide according to embodiment 50 comprising an amino acid sequence having at least 90% identity with the amino acid sequence in SEQ ID No. 6.

### EMBODIMENT 52

An isolated peptide according to embodiment 51 comprising an amino acid sequence having at least 95% identity with the amino acid sequence in SEQ ID No. 6.

### EMBODIMENT 53

An isolated peptide according to embodiment 52 comprising an amino acid sequence, which is as defined in SEQ ID No. 6.

### EMBODIMENT 54

A peptide with a sequence as defined in SEQ ID No. 1 comprising one or more of the substitutions Tyr-75 e acidic amino acid residue; Tyr-302→basic amino acid residue which is not Tyr; and Asp-304→basic amino acid residue.

### EMBODIMENT 55

A peptide according to embodiment 54 having a sequence as defined by SEQ ID No. 1 comprising one or more of the substitutions Tyr-75→Asp or Glu; Tyr-302→Arg or Lys; and Asp-304→Tyr, Lys or Arg.

### EMBODIMENT 56

A peptide according to embodiment 54 or embodiment 55 having a sequence as defined by SEQ ID No. 1 comprising one or more of the substitutions Tyr-75→Glu; Tyr-302→Arg; and Asp-304→Lys.

### EMBODIMENT 57

A peptide according to any of embodiments 54 to 56, wherein the sequence is as defined in SEQ ID No. 2.

### EMBODIMENT 58

A peptide according to any of embodiments 54 to 56, wherein the sequence is as defined in SEQ ID No. 3.

### EMBODIMENT 59

A peptide according to any of the embodiments 54 to 56, wherein the sequence is as defined in SEQ ID No. 4.

### EMBODIMENT 60

A peptide according to embodiment 54, wherein the sequence is as defined in SEQ ID No. 5.

### EMBODIMENT 61

A peptide according any of embodiments 54 to 60, wherein said peptide is an isolated peptide.

### EMBODIMENT 62

An isolated peptide according to any of embodiments 1 to 61, which peptide has transglutaminase activity.

### EMBODIMENT 63

An isolated peptide according to any of embodiments 1 to 62, which peptide has a specificity for Gln-40 of hGH compared to Gln-141 of hGH, which is different from the specificity of a peptide having an amino acid sequence as shown in SEQ ID No. 1 for Gln-40 of hGH compared to Gln-141 of hGH.

### EMBODIMENT 64

An isolated peptide according to embodiment 63, which peptide has a specificity for Gln-40 of hGH compared to Gln-141 of hGH, which is higher than the specificity of a peptide having an amino acid sequence as shown in SEQ ID No. 1 for Gln-40 of hGH compared to Gln-141 of hGH.

### EMBODIMENT 65

An isolated peptide according to embodiment 63, which peptide has a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

### EMBODIMENT 66

An isolated peptide according to embodiment 65, with the proviso that said modification does not solely consist of a substitution in Tyr-75 and/or a substitution in Tyr-302 and/or a substitution in Asp-304.

### EMBODIMENT 67

An isolated peptide according to embodiment 65, with the proviso that said modification does not solely consist of a substitution of Tyr-75 with an acidic amino acid residue, and/or a substitution in Tyr-302 and/or a substitution in Asp-304.

### EMBODIMENT 68

An isolated peptide according to embodiment 65, with the proviso that said modification does not solely consist of a substitution of Tyr-75 with Asp or Glu, and/or a substitution in Tyr-302 and/or a substitution in Asp-304.

### EMBODIMENT 69

An isolated peptide according to embodiment 65, with the proviso that said modification does not solely consist of a substitution of Tyr-75 with Glu, and/or a substitution in Tyr-302 and/or a substitution in Asp-304.

### EMBODIMENT 70

An isolated peptide according to embodiment 65, with the proviso that said modification does not solely consist of a substitution of Tyr-302 with a basic amino acid residue different from Tyr, and/or a substitution in Tyr-75 and/or a substitution in Asp-304.

### EMBODIMENT 71

An isolated peptide according to embodiment 65, with the proviso that said modification does not solely consist of a substitution of Tyr-302 with Arg or Lys, and/or a substitution in Tyr-75 and/or a substitution in Asp-304.

### EMBODIMENT 72

An isolated peptide according to embodiment 65, with the proviso that said modification does not solely consist of a substitution of Tyr-302 with Arg, and/or a substitution in Tyr-75 and/or a substitution in Asp-304.

### EMBODIMENT 73

An isolated peptide according to embodiment 65, with the proviso that said modification does not solely consist of a substitution of Asp-304 with a basic amino acid residue different from Tyr, and/or a substitution in Tyr-75 and/or a substitution in Tyr-302.

### EMBODIMENT 74

An isolated peptide according to embodiment 65, with the proviso that said modification does not solely consist of a substitution of Asp-304 with Tyr, Arg or Lys, and/or a substitution in Tyr-75 and/or a substitution in Tyr-302.

### EMBODIMENT 75

An isolated peptide according to embodiment 65, with the proviso that said modification does not solely consist of a substitution of Asp-304 with Lys, and/or a substitution in Tyr-75 and/or a substitution in Tyr-302.

### EMBODIMENT 76

An isolated peptide according to embodiment 65, with the proviso that said modified sequence is not SEQ ID No. 2, SEQ ID No. 3, SEQ ID No. 4 or SEQ ID No. 5.

### EMBODIMENT 77

An isolated peptide according to embodiment 65, with the proviso that said modification does not solely consist of a substitution of Tyr-75 with a acidic amino acid residue and/or a substitution of Tyr-302 with a basic amino acid residue, which is not Tyr; and/or a substitution of Asp-304 with a basic amino acid residue.

### EMBODIMENT 78

An isolated peptide according to embodiment 65, with the proviso that said modification does not solely consist of a substitution of Tyr-75 with Asp or Glu, and/or a substitution of Tyr-302 with Arg or Lys; and/or a substitution of Asp-304 with Tyr, Lys or Arg.

### EMBODIMENT 79

An isolated peptide according to embodiment 65, with the proviso that said modification does not solely consist of a substitution of Tyr-75 with Glu; and/or a substitution of Tyr-302 with Arg; and/or a substitution of Asp-304 with Lys.

### EMBODIMENT 80

An isolated peptide according to embodiment 65, with the proviso that said modification does not solely consist of a substitution of Tyr-75 with Glu and a substitution of Tyr-302 with Arg.

### EMBODIMENT 81

An isolated peptide according embodiment 65, with the proviso that said sequence does not comprise exactly one single mutation a compared to SEQ ID No. 1, which single mutation is a mutation in Tyr-75.

### EMBODIMENT 82

An isolated peptide according embodiment 65, with the proviso that said sequence does not comprise exactly one single mutation a compared to SEQ ID No. 1, which single mutation is Tyr-75 substituted with an acidic amino acid residue.

### EMBODIMENT 83

An isolated peptide according embodiment 65, with the proviso that said sequence does not comprise exactly one single mutation a compared to SEQ ID No. 1, which single mutation is Tyr-75 substituted with Asp or Glu.

### EMBODIMENT 84

An isolated peptide according embodiment 65, with the proviso that said sequence does not comprise exactly one single mutation a compared to SEQ ID No. 1, which single mutation is Tyr-75 substituted with Glu.

### EMBODIMENT 85

An isolated peptide according embodiment 65, with the proviso that said sequence does not comprise exactly one single mutation a compared to SEQ ID No. 1, which single mutation is a mutation in Tyr-302.

### EMBODIMENT 86

An isolated peptide according embodiment 65, with the proviso that said sequence does not comprise exactly one single mutation a compared to SEQ ID No. 1, which single mutation is Tyr-302 substituted with a basic amino acid residue different from Tyr.

### EMBODIMENT 87

An isolated peptide according embodiment 65, with the proviso that said sequence does not comprise exactly one single mutation a compared to SEQ ID No. 1, which single mutation is Tyr-302 substituted with Arg or Lys.

### EMBODIMENT 88

An isolated peptide according embodiment 65, with the proviso that said sequence does not comprise exactly one single mutation a compared to SEQ ID No. 1, which single mutation is Tyr-302 substituted with Arg.

### EMBODIMENT 89

An isolated peptide according embodiment 65, with the proviso that said sequence does not comprise exactly one single mutation a compared to SEQ ID No. 1, which single mutation is a mutation in Asp-304.

### EMBODIMENT 90

An isolated peptide according embodiment 65, with the proviso that said sequence does not comprise exactly one single mutation a compared to SEQ ID No. 1, which single mutation is Asp-304 substituted with a basic amino acid residue.

### EMBODIMENT 91

An isolated peptide according embodiment 65, with the proviso that said sequence does not comprise exactly one single mutation a compared to SEQ ID No. 1, which single mutation is Asp-304 substituted with Tyr, Arg or Lys.

### EMBODIMENT 92

An isolated peptide according embodiment 65, with the proviso that said sequence does not comprise exactly one single mutation a compared to SEQ ID No. 1, which single mutation is Asp-304 substituted with Lys.

### EMBODIMENT 93

An isolated peptide according to embodiment 65, with the proviso that said modification does not solely consist of a substitution in Asp-4 and/or a substitution in Val-30 and/or a substitution in Gly-250.

### EMBODIMENT 94

An isolated peptide according to embodiment 65, with the proviso that said modification does not solely consist of a substitution of Asp-4 with Glu and/or a substitution in Val-30 and/or a substitution in Gly-250.

### EMBODIMENT 95

An isolated peptide according to embodiment 65, with the proviso that said modification does not solely consist of a substitution of Val-30 with Ile and/or a substitution in Asp-4 and/or a substitution in Gly-250.

### EMBODIMENT 96

An isolated peptide according to embodiment 65, with the proviso that said modification does not solely consist of a substitution of Gly-250 with Thr and/or a substitution in Asp-4 and/or a substitution in Val-30.

### EMBODIMENT 97

An isolated peptide according to embodiment 65, with the proviso that said modification does not solely consist of a substitution of Gly-250 with Ser and/or a substitution in Asp-4 and/or a substitution in Val-30.

### EMBODIMENT 98

An isolated peptide according embodiment 65, with the proviso that said sequence does not comprise exactly one single mutation a compared to SEQ ID No. 1, which single mutation is a mutation in Asp-4.

### EMBODIMENT 99

An isolated peptide according embodiment 65, with the proviso that said sequence does not comprise exactly one single mutation a compared to SEQ ID No. 1, which single mutation is Asp-4 substituted with Glu.

### EMBODIMENT 100

An isolated peptide according embodiment 65, with the proviso that said sequence does not comprise exactly one single mutation a compared to SEQ ID No. 1, which single mutation is a mutation in Val-30.

### EMBODIMENT 101

An isolated peptide according embodiment 65, with the proviso that said sequence does not comprise exactly one single mutation a compared to SEQ ID No. 1, which single mutation is Val-30 substituted with Ile.

### EMBODIMENT 102

An isolated peptide according embodiment 65, with the proviso that said sequence does not comprise exactly one single mutation a compared to SEQ ID No. 1, which single mutation is a mutation in Gly-250.

### EMBODIMENT 103

An isolated peptide according embodiment 65, with the proviso that said sequence does not comprise exactly one single mutation a compared to SEQ ID No. 1, which single mutation is Gly-250 substituted with Thr.

### EMBODIMENT 104

An isolated peptide according embodiment 65, with the proviso that said sequence does not comprise exactly one single mutation a compared to SEQ ID No. 1, which single mutation is Gly-250 substituted with Ser.

### EMBODIMENT 105

An isolated peptide according embodiment 65, with the proviso that said sequence does not comprise exactly one single mutation a compared to SEQ ID No. 1, which single mutation is a mutation in Asp-4.

### EMBODIMENT 106

An isolated peptide according embodiment 65, with the proviso that said sequence does not comprise exactly one single mutation a compared to SEQ ID No. 1, which single mutation is Asp-4 substituted with Glu.

### EMBODIMENT 107

An isolated peptide according embodiment 65, with the proviso that said sequence does not comprise exactly one single mutation a compared to SEQ ID No. 1, which single mutation is a mutation in Val-30.

### EMBODIMENT 108

An isolated peptide according embodiment 65, with the proviso that said sequence does not comprise exactly one single mutation a compared to SEQ ID No. 1, which single mutation is Val-30 substituted with Ile.

### EMBODIMENT 109

A transglutaminase peptide having a specificity for Gln-40 of hGH compared to Gln-141 of hGH, which is different from the specificity of a peptide having an amino acid sequence as shown in SEQ ID No. 1 for Gln-40 of hGH compared to Gln-141 of hGH.

### EMBODIMENT 110

A transglutaminase peptide according to embodiment 109 having a specificity for Gln-40 of hGH compared to Gln-141 of hGH, which is higher than the specificity of a peptide having an amino acid sequence as shown in SEQ ID No. 1 for Gln-40 of hGH compared to Gln-141 of hGH.

### EMBODIMENT 111

A transglutaminase peptide according to embodiment 109 having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

### EMBODIMENT 112

A nucleic acid construct encoding a peptide according to any of embodiments 1 to 111.

### EMBODIMENT 113

A vector comprising the nucleic acid construct of embodiment 112.

### EMBODIMENT 114

A host comprising the vector of embodiment 113.

### EMBODIMENT 115

A composition comprising a peptide according to any of embodiments 1 to 111.

### EMBODIMENT 116

A method for conjugating hGH, wherein said method comprises reacting said hGH with an amine donor in the presence of a peptide according to any of embodiments 1 to 111.

### EMBODIMENT 117

A method for conjugating hGH according to embodiment 116, wherein the amount of hGH conjugated at position Gln-40 as compared to the amount of hGH conjugated at position Gln-141 is significantly increased in comparison with the amount of hGH conjugated at position Gln-40 as compared to the amount of hGH conjugated at position Gln-141 when a peptide having the amino acid sequence as shown in SEQ ID No. 1 is used in said method instead of the peptide according to any of embodiments 1 to 111.

### EMBODIMENT 118

A method for conjugating hGH according to embodiment 116, wherein the amount of hGH conjugated at position Gln-141 as compared to the amount of hGH conjugated at position Gln-40 is significantly increased in comparison with the amount of hGH conjugated at position Gln-141 as compared to the amount of hGH conjugated at position Gln-40, when a peptide having the amino acid sequence as shown in SEQ ID No. 1 is used in said method instead of the peptide according to any of embodiments 1 to 111.

### EMBODIMENT 119

Use of a peptide according to any of embodiments 1 to 111 in the preparation of a conjugated hGH.

### EMBODIMENT 120

Use according to embodiment 119, wherein the hGH is conjugated in position Gln-40.

### EMBODIMENT 121

Use according to embodiment 119, wherein the hGH is conjugated in position Gln-141.

If a TGase as defined in SEQ ID No. 2 is used, predominantly Gln-141 functionalised hGH is obtained and only negligible amounts of Gln-40 functionalised or Gln-40/Gln-141 double-functionalised hGH is obtained.

In one embodiment, the invention relates to a peptide comprising an amino acid sequence as defined in SEQ ID No. 1, in which sequence Tyr-75 has been substituted with Asp or Glu; and/or Tyr-302 has been substituted with Arg or Lys; and/or Asp-304 has been substituted with Tyr, Lys or Arg and/or Gly-250 has been substituted with Ser or Thr; and/or Asp-4 has been substituted with Glu; and/or Val-30 has been substituted with Ile; and/or amino acids. 14 has been deleted and replaced with Gly (Δ(DSDD)1-4G).

In one embodiment, the invention relates to a peptide comprising an amino acid sequence as defined in SEQ ID No. 1, wherein Tyr-75 has been substituted with Glu; and/or Tyr-302 has been substituted with Arg; and/or Asp-304 has been substituted with Lys; and/or Gly-250 has been substituted with Ser; and/or Asp-4 has been substituted with Glu; and/or Val-30 has been substituted with Ile; and/or amino acids 14 has been deleted.

In one embodiment, the invention relates to a peptide comprising an amino acid sequence as defined in SEQ ID No. 2, which is SEQ ID No. 1 with a Tyr-754→Glu substitution:

In one embodiment, the invention relates to a peptide comprising an amino acid sequence as defined in SEQ ID No. 3, which is SEQ ID No. 1 with a Tyr-304→Arg substitution.

In one embodiment, the invention relates to a peptide comprising an amino acid sequence as defined in SEQ ID No. 4, which is SEQ ID No. 1 with a Asp-304→Lys substitution.

In one embodiment, the invention relates to a peptide comprising an amino acid sequence as defined in SEQ ID No. 5, which is SEQ ID No. 1 with a Tyr-75→Glu substitution, a Tyr-302→Arg substitution, and a Asp-304→Lys substitution.

In one embodiment, the invention relates to a peptide comprising an amino acid sequence as defined in SEQ ID No. 6, which is the TGase of Streptoverticillium ladakanum.

The peptides of the present invention exhibit TGase activity as determined in the assay described in U.S. Pat. No. 5,156,956. Briefly described, the measurement of the activity of a given peptide is carried out by performing a reaction using benzyloxycarbonyl-L-glutaminyl glycine and hydroxylamine as substrates in the absence of Ca²⁺, forming an iron complex with the resulting hydroxamic acid in the presence of trichloroacetic acid, measuring absorption at 525 nm and determining the amount of hydroxamic acid by a calibration curve to calculate the activity. For the purpose of this specification, an peptide, which exhibits transglutaminase activity in said assay is deemed to be have transglutaminase activity. In particular, the TGase variants of the present invention exhibit an activity which is more than 30%, such as more than 50%, such as more than 70%, such as more than 90% of that of TGase from S. mobaraense.

In one embodiment, the invention relates to a composition comprising a polypeptide having any of SEQ ID No.'s: 2, 3, 4, or 5.

The peptides of the present invention may be prepared in different ways. The peptides may be prepared by protein synthetic methods known in the art. Due to the size of the peptides, this may be done more conveniently by synthesising several fragments of the peptides which are then combined to provide the peptides of the present invention. In a particular embodiment, however, the peptides of the present invention are prepared by fermentation of a suitable host comprising a nucleic acid construct encoding the peptides of the present invention.

In one embodiment, the invention also relates to nucleic acid constructs encoding the peptides of the present invention.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a protein of interest. The construct may optionally contain other nucleic acid segments.

The nucleic acid construct of the invention encoding the peptide of the invention may suitably be of genomic or cDNA origin, for instance obtained by preparing a genomic or cDNA library and screening for DNA sequences coding for all or part of the protein by hybridization using synthetic oligonucleotide probes in accordance with standard techniques (cf. J. Sambrook et al, 1989, Molecular Cloning, A Laboratory Manual, 2d edition, Cold Spring Harbor, N.Y.) and by introducing the mutations as it is known in the art.

The nucleic acid construct of the invention encoding the protein may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239, 487-491 (1988).

The nucleic acid construct is preferably a DNA construct which term will be used exclusively in the following.

In a further aspect, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the protein of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the protein.

The promoter may be any DNA sequence which shows transcriptional activity in the host cell of choice and may be derived from genes encoding proteins either homologous or heterologous to the host cell.

Examples of suitable promoters for use in yeast host cells include promoters from yeast glycolytic genes (Hitzeman et al., J. Biol. Chem. 255, 12073-12080 (1980); Alber and Kawasaki, J. Mol. Appl. Gen. 1, 419-434 (1982)) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4-c (Russell et al., Nature 304, 652-654 (1983)) promoters.

Examples of suitable promoters for use in filamentous fungus host cells are, for instance, the ADH3 promoter (McKnight et al., The EMBO J. 4, 2093-2099 (1985)) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizo-mucor miehei lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding the protein of the invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question.

When the host cell is a yeast cell, suitable sequences enabling the vector to replicate are the yeast plasmid 2μ replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 125-130 (1985)), or one which confers resistance to a drug, e.g. ampicillin, kanamycin, tetracyclin, chloramphenicol, neomycin, hygromycin or methotrexate. For filamentous fungi, selectable markers include amdS, pvrG, argB, niaD and sC.

To direct a protein of the present invention into the secretory pathway of the host cells, a secretory signal sequence (also known as a leader sequence, prepro sequence or pre sequence) may be provided in the recombinant vector. The secretory signal sequence is joined to the DNA sequence encoding the protein in the correct reading frame. Secretory signal sequences are commonly positioned 5′ to the DNA sequence encoding the protein. The secretory signal sequence may be that normally associated with the protein or may be from a gene encoding another secreted protein.

For secretion from yeast cells, the secretory signal sequence may encode any signal peptide which ensures efficient direction of the expressed protein into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 643-646 (1981)), a modified carboxypeptidase signal peptide (cf. L. A. Valls et al., Cell 48, 887-897 (1987)), the yeast BAR1 signal peptide (cf. WO 87/02670), or the yeast aspartic protease 3 (YAP3) signal peptide (cf. M. Egel-Mitani et al., Yeast 6, 127-137 (1990)).

For efficient secretion in yeast, a sequence encoding a leader peptide may also be inserted downstream of the signal sequence and upstream of the DNA sequence encoding the protein. The function of the leader peptide is to allow the expressed protein to be directed from the endoplasmic reticulum to the Golgi apparatus and further to a secretory vesicle for secretion into the culture medium (i.e. exportation of the protein across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase.

The procedures used to ligate the DNA sequences coding for the present protein, the promoter and optionally the terminator and/or secretory signal sequence, respectively, and to insert them into suitable vectors containing the information necessary for replication, are well known to persons skilled in the art (cf., for instance, Sambrook et al., op. cit.).

The host cell into which the DNA construct or the recombinant vector of the invention is introduced may be any cell which is capable of producing the present protein and includes bacteria, yeast, fungi and higher eukaryotic cells.

Examples of bacterial host cells which, on cultivation, are capable of producing the protein of the invention are grampositive bacteria such as strains of Bacillus, such as strains of B. subtilis, B. lichenifonnis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gramnegative bacteria such as Echerichia coli. The transformation of the bacteria may be effected by protoplast transformation or by using competent cells in a manner known per se (cf. Sambrook et al., supra). Other suitable hosts include S. mobaraense, S. lividans, and C. glutamicum (Appl. Microbiol. Biotechnol. 64, 447-454 (2004)).

When expressing the protein in bacteria such as E. coli, the protein may be retained in the cytoplasm, typically as insoluble granules (known as inclusion bodies), or may be directed to the periplasmic space by a bacterial secretion sequence. In the former case, the cells are lysed and the granules are recovered and denatured after which the protein is refolded by diluting the denaturing agent. In the latter case, the protein may be recovered from the periplasmic space by disrupting the cells, e.g. by sonication or osmotic shock, to release the contents of the periplasmic space and recovering the protein.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 3459-3465 (1986); U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al. Gene 78, 147-156 (1989).

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

The transformed or transfected host cell described above is then cultured in a suitable nutrient medium under conditions permitting the expression of the present peptide, after which the resulting protein is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The protein produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of protein in question.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### EXAMPLES

### Example 1

### PEGylation of hGH

a) hGH is dissolved in phosphate buffer (50 mM, pH 8.0). This solution is mixed with a solution of amine donor, e.g. 1,3-diamino-propan-2-ol dissolved in phosphate buffer (50 mM, 1 ml, pH 8.0, pH adjusted to 8.0 with dilute hydrochloric acid after dissolution of the amine donor).
   Finally a solution of TGase (˜40 U) dissolved in phosphate buffer (50 mM, pH 8.0, 1 ml) is added and the volume is adjusted to 10 ml by addition of phosphate buffer (50 mM, pH 8). The combined mixture is incubated for approximately 4 hours at 37° C. The temperature is lowered to room temperature and N-ethyl-maleimide (TGase inhibitor) is added to a final concentration of 1 mM. After further 1 hour the mixture is diluted with 10 volumes of tris buffer (50 mM, pH 8.5).
b) The transaminated hGH obtained from a) may then optionally be further reacted to activate a latent functional group if present in the amine donor.
c) The functionalised hGH obtained from a) or b) is then reacted with a suitably functionalised PEG capable of reacting with the functional group introduced into hGH. As an example, an oxime bond may be formed by reacting a carbonyl moiety (aldehyde or ketone) with an alkoxyamine.

### Example 2

### TGase Specificity Assay I

The method described may be used to determine the Gln residue(s) in the hGH, which has been modified in a reaction as described in Example 1. That is to say the method described here may be used to determine the selectivity of the TGase's of the present invention.

Determination of PEGylation Site(s)

Mono PEGylated hGH obtained in Example 1 is purified using a combination of ion-exchange chromatography and gel filtration.

In order to determine the site(s) of PEGylation the purified compounds are reduced and alkylated using dithiothreitol and iodoacetamide. Subsequently the compounds are digested using an un-specific protease, Proteinase K, and the resulting digest is separated on a reverse phase C-18 HPLC column using an acetonitrile/TFA buffer system. PEGylated peptides will under these conditions elute significantly later than un-PEGylated peptides and furthermore all PEGylated peptides (if there is more than one) will elute in the same peak, as the retention time of PEGylated peptides is mainly determined by the PEG-moiety.

The peak containing PEGylated peptides is collected and subjected to amino acid sequencing using automated Edman analysis. The results provide information both on the exact site of PEGylation—a PEGylated amino acid will produce a blank cycle in the sequencing analysis—and simultaneously on the number and relative amount of peptides present and thus reveal if PEGylation has taken place at more than one site.

### Example 3

### Testing of TGase Mutants for their Specificity Towards Gln-141 vs Gln-40 in hGH (Assay I)

20 μl 1,3-diamino-2-propanol (180 mg/ml in 10 mM phosphate buffer, pH adjusted to 8.3 by addition of concentrated HCl) is added to a solution of hGH (1 mg) in solution in 10 mM phosphate buffer pH 8.1 (50 μl). 10 mM phosphate buffer is added in an amount such that the final reaction mixture volume is 100 μl. The reaction is started by addition of the enzyme (final concentration 0.07 to 7 μM). The reaction mixture is incubated at 37° C., and the reaction followed by capillary electrophoresis (CE).

To an aliquot of the reaction mixture (10 μl) is added N-ethylmaleimide 100 mM (1 μl). The mixture is incubated at ambient temperature for 5 min, and then diluted 100 times in H₂O before CE analysis.

CE is carried out using an Agilent Technologies 3D-CE system (Agilent Technologies). Data acquisition and signal processing are performed using Agilent Technologies 3DCE ChemStation. The capillary is a 64.5 cm (56.0 cm efficient length) 50 μm i.d. “Extended Light Path Capillary” from Agilent. UV detection is performed at 200 nm (16 nm Bw, Reference 380 nm and 50 nm Bw). The running electrolyte is phosphate buffer 50 mM pH 7.0. The capillary is conditioned with 0.1 M NaOH for 3 min, then with Milli-Q water for 2 min and with the electrolyte for 3 min.

After each run, the capillary is flushed with milli-Q water for 2 min, then with phosphoric acid for 2 min, and with milli-Q water for 2 min. The hydrodynamic injection is done at 50 mbar for 4.0 s. The voltage is +25 kV. The capillary temperature is 30° C. and the runtime is 10.5 min.

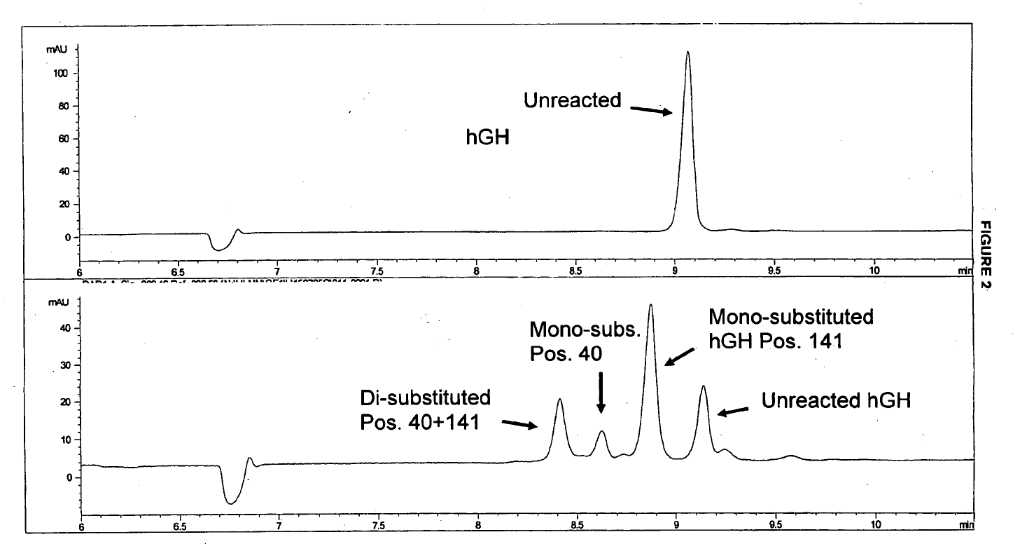

FIG. 2 shows a picture of a typical CE analysis of a TGase-catalyzed transglutamination of hGH with 1,3-diamino-2-propanol.

The enzyme amounts were adjusted so that the amounts of mono-transamination products reached their maximum within 5 h reaction time.

An indication of the reaction rates is given by the time at which half of the substrate hGH has been transaminated.

Table 1 shows the results for selected TGases

TABLE 1

| | | | About 50% hGH |
|---|---|---|---|
| | | Transamination | reacted at |
| Enzyme mutant | Concentration | Gln 40 vs Gln 141 | t= |
| WT | 0.07 μM | 15:85 | 1 h 15 |
| Y75E | 3.90 μM | 45:55 | <30 min |
| Y302R | 0.39 μM | 30:70 | 30 min |
| Y75E, Y302R | 7.20 μM | 65:35 | 1 h 30 |

WT TGase having the amino acid sequence of SEQ ID No. 1
Y75E TGase having the amino acid sequence of SEQ ID No. 2
Y302R TGase having the amino acid sequence of SEQ ID No. 3
Y75E, Y302R TGase as defined in SEQ ID No. 1, wherein Tyr-75 has been substituted with Glu and Tyr-302 has been substituted with Arg

### Example 4

### Testing of TGase Mutants for their Selectivity Towards Gln-141 vs Gln-40 in hGH (Assay II)

This assay uses two hGH mutants each having an asparagine residue instead of a glutamine at one of positions Gln-40 and Gln-141, leaving only one glutamine to react. The preparation of said mutants are described in Kunkel T A et al., Methods in Enzymology 154, 367-382 (1987), and Chung Nan Chang et al., Cell 55, 189-196 (1987). The hGH mutant Q40N is a model substrate for Gln-141 in hGH, and Q141N is a model substrate for Gln-40.

To 400 μl of buffer solution with 225 mM 1,3-diamino-2-propanol and 35 mM Tris (pH has been adjusted to 8.0 by addition of concentrated HCl), 600 μl of mutant hGH (1.5 mg/ml) and 5 μl of TGase (1.6 mg/ml) are added, The reaction mixture is incubated for 30 minutes at 25° C.

The subsequent analysis is performed by FPLC using a Mono Q 5/5 GL 1 ml (GE Health) column and UV detection at 280 nm. Buffer A: 20 mM triethanolamine pH 8.5; Buffer B: 20 mM triethanolamine 0.2 M NaCl pH 8.5; flow rate: 0.8 ml/min. The elution gradient is defined as following:

| Step | Time/min | % A | % B |
|---|---|---|---|
| 1 | 2.00 | 100.0 | 0.0 |
| 2 | 4.00 | 70.0 | 30.0 |
| 3 | 5.00 | 70.0 | 30.0 |
| 4 | 35.00 | 50.0 | 50.0 |

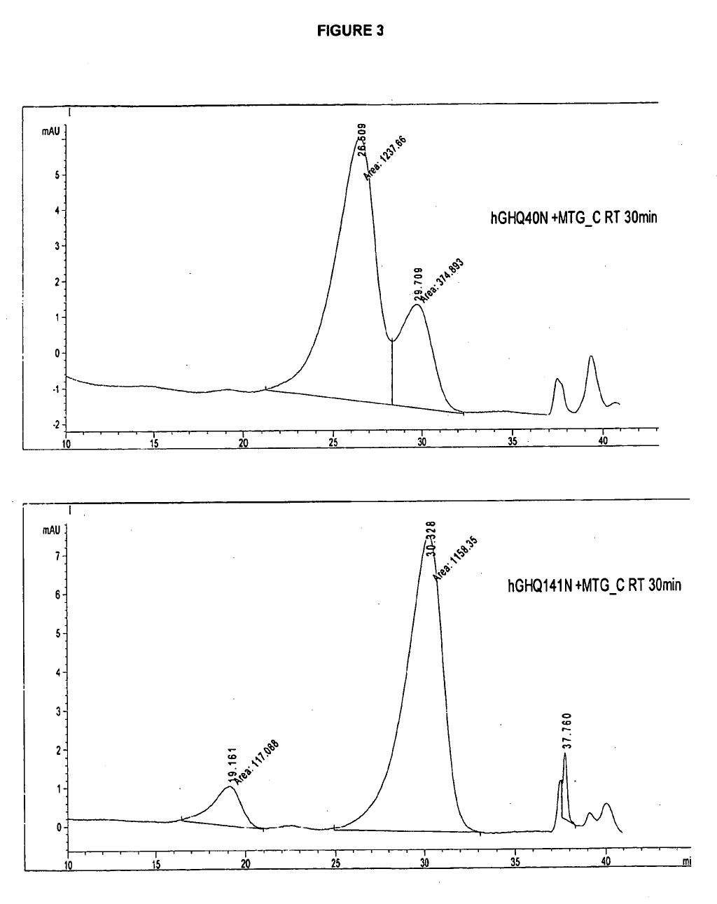

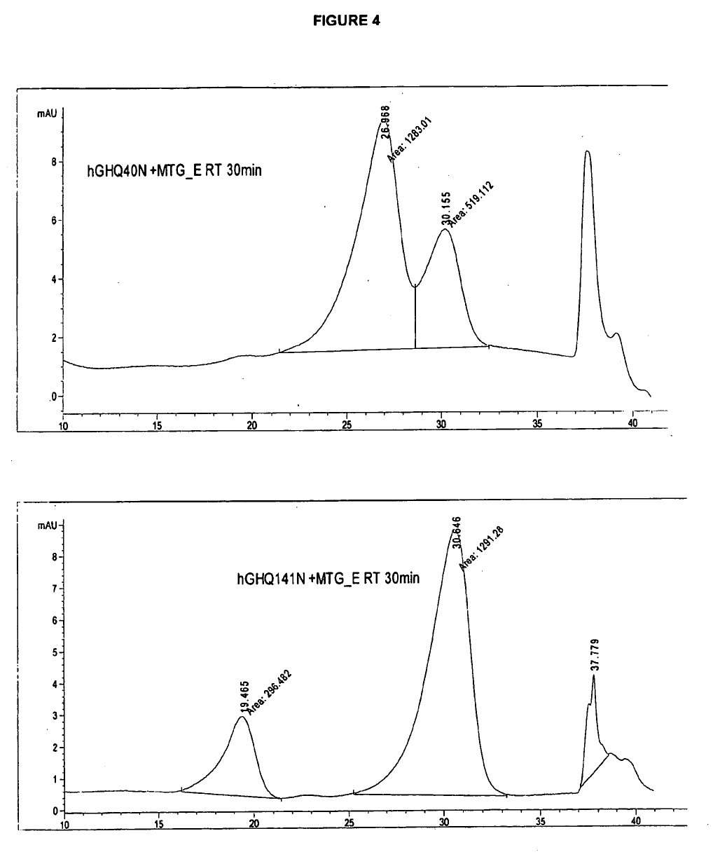

The selectivity ratio is then calculated from the ratio of the two areas (in arbitrary units) under the curves (shown in FIGS. 3 and 4) attributed to the two products, Q141 and Q40. The result achieved when using TGase from S. mobarense (SEQ ID No. 1) and S. ladakanum (SEQ ID No. 6) is shown in Table 2. Q40N+its product-Q141=Q141N+its product-Q40, and are normalized to 100.

TABLE 2

| | | product- | | product- | Transamination | Gln 40 vs Gln 141 |
|---|---|---|---|---|---|---|
| Enzyme | Q40N | Q141 | Q141N | Q40 | Gln 40 vs Gln 141 | (normalized) |
| mobarense | 29 | 71. | 81 | 19 | 19:71 | 21:79 |
| ladakanum | 23 | 77 | 90 | 10 | 10:77 | 11:89 |

1. An isolated peptide comprising an amino acid sequence having at least 80% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

2. An isolated peptide according to claim 1 comprising an amino acid sequence having at least 85% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

3. An isolated peptide according to claim 2 comprising an amino acid sequence having at least 90% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

4. An isolated peptide according to claim 3 comprising an amino acid sequence having at least 95% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

5. An isolated peptide according to claim 4 comprising an amino acid sequence as defined in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

6. An isolated peptide according to claim 1, wherein said sequence is modified in Gly-250.

7. An isolated peptide according to claim 6, wherein Gly-250 is substituted with a Thr.

8. An isolated peptide according to claim 6, wherein Gly-250 is substituted with a Ser.

9. An isolated peptide according to claim 1, wherein said sequence is modified in one or more of the amino acid residues situated less than 20 Å away from Cys-64.

10. An isolated peptide according to claim 1, wherein said sequence is modified in one or more of the amino acid residues situated less than 15 Å away from Cys-64.

11. An isolated peptide according to claim 10, wherein the sequence is not modified in position Cys64.

12. An isolated peptide according to claim 10, wherein said sequence is modified in one or more of the amino acid residues selected from Val-30, Tyr-62, Val-252, Asn-253, Phe-254, His-277, Tyr-278, and Leu-285.

13. An isolated peptide according to claim 12, wherein said sequence is modified in Tyr-62.

14. An isolated peptide according to claim 12, wherein said sequence is modified in His-277 and/or Tyr-278.

15. An isolated peptide according to claim 12, wherein said sequence is modified in Leu-285.

16. An isolated peptide according to claim 12, wherein said sequence is modified in one or more of the amino acid residues selected from Val-252, Asn-253, and Phe-254.

17. An isolated peptide according to claim 10, wherein said sequence is modified in Val-30.

18. An isolated peptide according to claim 17, wherein Val-30 is substituted with an Ile.

19. An isolated peptide according to claim 1, wherein said sequence is modified in one or more of the amino acid residues selected from Asp-4, Arg-89, Glu-115, Ser-210, Asp-221, and Lys-327.

20. An isolated peptide according to claim 19, wherein Asp-4 is substituted with an Glu.

21. An isolated peptide according to claim 19, wherein Asp-4 is substituted with an Glu and the amino acids in positions 1, 2 and 3 have been deleted.

22. An isolated peptide according to claim 19, wherein Arg-89 is substituted with a Lys.

23. An isolated peptide according to claim 19, wherein Glu-115 is substituted with an Asp.

24. An isolated peptide according to claim 19, wherein Ser-210 is substituted with a Gly.

25. An isolated peptide according to claim 19, wherein Asp-221 is substituted with a Ser.

26. An isolated peptide according to claim 19, wherein Lys-327 is substituted with a Thr.

27. An isolated peptide according to claim 1, wherein said sequence is modified in one or more of the amino acid residues selected from Ala-226 and Pro-227.

28. An isolated peptide according to claim 27, wherein Ala-226 is substituted with an Asp.

29. An isolated peptide according to claim 27, wherein Pro-227 is substituted with an Arg.

30. An isolated peptide according to claim 1, wherein said sequence is modified in Tyr-75.

31. An isolated peptide according to claim 30, wherein Tyr-75 is substituted with an amino acid different from Glu.

32. An isolated peptide according to claim 31, wherein Tyr-75 is substituted with an amino acid different from Asp or Glu.

33. An isolated peptide according to claim 32, wherein Tyr-75 is substituted with an amino acid different from an acidic amino acid residue.

34. An isolated peptide according to claim 30, wherein Tyr-75 is substituted with an acidic amino acid residue.

35. An isolated peptide according to claim 34, wherein Tyr-75 is substituted with Asp or Glu.

36. An isolated peptide according to claim 35, wherein Tyr-75 is substituted with Glu.

37. An isolated peptide according to claim 1, wherein said sequence is modified in Tyr-302.

38. An isolated peptide according to claim 37, wherein Tyr-302 is substituted with a basic amino acid residue different from Tyr.

39. An isolated peptide according to claim 38, wherein Tyr-302 is substituted with Arg or Lys.

40. An isolated peptide according to claim 39, wherein Tyr-302 is substituted with Arg.

41. An isolated peptide according to claim 1, wherein said sequence is modified in Asp-304.

42. An isolated peptide according to claim 41, wherein Asp-304 is substituted with a basic amino acid residue.

43. An isolated peptide according to claim 42, wherein Asp-304 is substituted with Tyr, Lys or Arg.

44. An isolated peptide according to claim 43, wherein Asp-304 is substituted with Lys.

45. An isolated peptide according to claim 36 having a sequence as defined in SEQ ID No. 2.

46. An isolated peptide according to claim 40 having a sequence as defined in SEQ ID No. 3.

47. An isolated peptide according to claim 44 having a sequence as defined in SEQ ID No. 4.

48. An isolated peptide according to claim 30 having a sequence as defined in SEQ ID No. 5.

49. An isolated peptide comprising an amino acid sequence having at least 80% identity with the amino acid sequence in SEQ ID No. 6.

50. An isolated peptide according to claim 49 comprising an amino acid sequence having at least 85% identity with the amino acid sequence in SEQ ID No. 6.

51. An isolated peptide according to claim 50 comprising an amino acid sequence having at least 90% identity with the amino acid sequence in SEQ ID No. 6.

52. An isolated peptide according to claim 51 comprising an amino acid sequence having at least 95% identity with the amino acid sequence in SEQ ID No. 6.

53. An isolated peptide according to claim 52 comprising an amino acid sequence, which is as defined in SEQ ID No. 6.

54. A peptide with a sequence as defined in SEQ ID No. 1 comprising one or more of the following substitutions Tyr-75→to an acidic amino acid residue; Tyr-302→to a basic amino acid residue which is not Tyr; and Asp-304→to a basic amino acid residue.

55. A peptide according to claim 54 having a sequence as defined by SEQ ID No. 1 comprising one or more of the following substitutions: Tyr-75 to Asp or Glu; Tyr-302→to Arg or Lys; and Asp-304→to Tyr, Lys or Arg.

56. A peptide according to claim 54 having a sequence as defined by SEQ ID No. 1 comprising one or more of the following substitutions Tyr-75→to Glu; Tyr-302→to Arg; and Asp-304→to Lys.

57. A peptide according to claim 54, wherein the sequence is as defined in SEQ ID No. 2.

58. A peptide according to claim 54, wherein the sequence is as defined in SEQ ID No. 3.

59. A peptide according to claim 54, wherein the sequence is as defined in SEQ ID No. 4.

60. A peptide according to claim 54, wherein the sequence is as defined in SEQ ID No. 5.

61. A peptide according to claim 54, wherein said peptide is an isolated peptide.

62. A peptide according to claim 1, which peptide has transglutaminase activity.

63. A peptide according to claim 1, which peptide has a specificity for Gln-40 of hGH compared to Gln-141 of hGH, which is different from the specificity of a peptide having an amino acid sequence as shown in SEQ ID No. 1 for Gln-40 of hGH compared to Gln-141 of hGH.

64. An isolated peptide according to claim 63, which peptide has a specificity for Gln-40 of hGH compared to Gln-141 of hGH, which is higher than the specificity of a peptide having an amino acid sequence as shown in SEQ ID No. 1 for Gln-40 of hGH compared to Gln-141 of hGH.

65. An isolated peptide according to claim 63, which peptide has a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

66. A transglutaminase peptide having a specificity for Gln-40 of hGH compared to Gln-141 of hGH, which is different from the specificity of a peptide having an amino acid sequence as shown in SEQ ID No. 1 for Gln-40 of hGH compared to Gln-141 of hGH.

67. A transglutaminase peptide according to claim 66 having a specificity for Gln-40 of hGH compared to Gln-141 of hGH, which is higher than the specificity of a peptide having an amino acid sequence as shown in SEQ ID No. 1 for Gln-40 of hGH compared to Gln-141 of hGH.

68. A transglutaminase peptide according to claim 66 having a specificity for Gln-141 of hGH compared to Gln-40 of hGH, which is higher than the specificity of a peptide having an amino acid sequence as shown in SEQ ID No. 1 for Gln-141 of hGH compared to Gln-40 of hGH.

69. A nucleic acid construct encoding a peptide according to claim 1.

70. A vector comprising the nucleic acid construct of claim 69.

71. A host comprising the vector of claim 70.

72. A composition comprising a peptide comprising an amino acid sequence having at least 80% identity with the amino acid sequence in SEQ ID No. 1, wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

73. A method for conjugating hGH, wherein said method comprises reacting said hGH with an amine donor in the presence of a peptide comprising an amino acid sequence having at least 80% identity with the amino acid sequence in SEQ ID No. 11 wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

74. A method for conjugating hGH according to claim 73, wherein the amount of hGH conjugated at position Gln-40 as compared to the amount of hGH conjugated at position Gln-141 is significantly increased in comparison with the amount of hGH conjugated at position Gln-40 as compared to the amount of hGH conjugated at position Gln-141 when a peptide having the amino acid sequence as shown in SEQ ID No. 1 is used in said method instead of the peptide comprising an amino acid sequence having at least 80% identity with the amino acid sequence in SEQ ID No. 1 wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-250, Val-252, Asn-253, Phe-254, His-277, Tyr-278, Leu-285, Tyr-302, Asp-304, and Lys-327.

75. A method for conjugating hGH according to claim 73, wherein the amount of hGH conjugated at position Gln-141 as compared to the amount of hGH conjugated at position Gln-40 is significantly increased in comparison with the amount of hGH conjugated at position Gln-141 as compared to the amount of hGH conjugated at position Gln-40, when a peptide having the amino acid sequence as shown in SEQ ID No. 1 is used in said method instead of the peptide comprising an amino acid sequence having at least 80% identity with the amino acid sequence in SEQ ID No. 1 wherein said sequence is modified in one or more of the amino acid residues selected from Asp-4, Val-30, Tyr-62, Tyr-75, Arg-89, Glu-115, Ser-210, Asp-221, Ala-226, Pro-227, Gly-2501 Val-252, Asn-253, Phe-254, His-277, Tyr-278, Leu-285, Tyr-302, Asp-304, and Lys-327.

76. (canceled)

77. (canceled)

78. (canceled)


SEQUENCE LISTING (6 sequences)

SEQ ID NO: 1 - protein, 331 aa, Streptoverticillium mobaraense
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 2 - protein, 331 aa, Artificial sequence
  Streptoverticillium mobaraense variant
DSDDRVTPPAEPLDRMPDPYRPSYGRAETVVNNYIRKWQQVYSHRDGRKQQMTEEQREWLSYGCVGVTWVNSGQEPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 3 - protein, 331 aa, Artificial sequence
  Streptoverticillium mobaraense variant
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGRSDFDRGAYVITFIPKSWNTAPDKVKQGWP

SEQ ID NO: 4 - protein, 331 aa, Artificial sequence
  Streptoverticillium mobaraense variant
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSKFDRGAYVITFIPKSWNTAPDKVKQGWP

SEQ ID NO: 5 - protein, 331 aa, Artificial sequence
  Streptoverticillium mobaraense variant
DSDDRVTPPAEPLDRMPDPYRPSYGRAETVVNNYIRKWQQVYSHRDGRKQQMTEEQREWLSYGCVGVTWVNSGQEPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGRSKFDRGAYVITFIPKSWNTAPDKVKQGWP

SEQ ID NO: 6 - protein, 331 aa, Streptoverticillium ladakanum
DSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVKQGWP